(19) United States
(12) Patent Application Publication
(10) Pub. No.: US 2015/0025352 A1
(43) Pub. Date: Jan. 22, 2015

(54) METHOD AND DEVICE FOR DETERMINING BRAIN AND SCALP STATE

(71) Applicant: NorDocs Technologies Inc., Ottawa (CA)

(72) Inventors: Herschel B. Caytak, Ottawa (CA); Abeye Mekonnen, Ottawa (CA); Izmail Batkin, Ottawa (CA)

(73) Assignee: NorDocs Technologies Inc., Ottawa (CA)

(21) Appl. No.: 13/947,920

(22) Filed: Jul. 22, 2013

(51) Int. Cl. A61B 5/053; A61B 5/00

(52) U.S. Cl. CPC A61B 5/0531; A61B 5/6814; A61B 5/7282; A61B 5/7246; A61B 5/4064
USPC 600/383; 600/547

(57) ABSTRACT

A method for determining brain state has the steps of attaching at least 2 first electrodes to the scalp of a subject at a separation, transmitting an electrical current between the at least 2 first electrodes, and measuring a first impedance between the at least 2 first electrodes to provide a resulting impedance. One embodiment has the further step of comparing the resulting impedance to pre-stored impedance measurements of the scalp to determine normal or pathological state. A further embodiment has the steps of attaching 2 first electrodes to the scalp of a subject at a separation and 2 second electrodes, transmitting a current between the first electrodes and measuring a first impedance between the second electrodes to provide a resulting impedance. The electrodes may be driven shield electrodes, and the method may further include comparing impedance measurements to pre-stored impedance measurements to determine normal and pathological state.

### BACKGROUND OF THE INVENTION

1. Field of the Invention

The invention relates to a method for determining brain and scalp state, as well as a device for determining brain and scalp state.

2. Description of the Prior Art

Human tissues are known to have electrical properties i.e. they are able to conduct electricity as well as be polarized by an externally applied electric field. The term dielectric is reserved to describe electrical properties of polarisable materials. Thus, we refer to the properties of tissues as dielectric herein. Impedance measurements and hence dielectric properties can be taken either at a single or multiple frequency probe current and can be useful physical quantities to detect abnormalities as well as tissue classification. Biological tissues' dielectric properties exhibit strong dependence on the frequency of the probe current and changes to tissue composition and structure can be detected through a spectroscopic sweep of the tissue dielectric properties. The electrical impedance may be calculated for each frequency whereas the real aspect of the impedance is represented by the purely resistive component, and the imaginary aspect of the impedance is represented by the capacitive reactance component. One possible method of determining tissue pathological state is by analysis of the profile and shifts of the resistance and reactance curves over the frequency spectrum although other methods may be possible.

Impedance measurements are being developed for use for a range of health related applications including monitoring ischemia, hypoxia, stroke related bleeding, subdural hematoma and diagnosing cirrhosis of the liver, liver cancer, lung cancer, and breast cancer. A hitherto unexplored application is using impedance measurements to detect abnormal brain state characteristic of addiction. Various forms of addiction including substance abuse and behavioral addictions cause structural and physiological changes in the brain tissues. For instance, reduced gray matter volume is reported in substance abusers of cocaine and heroin; amphetamine type stimulants; nicotine and alcohol addicts as compared to normal controls. Structural damage to the integrity and tract coherence of the white matter fibers has also been reported for a wide range of addictions. Furthermore, physiological functions in the cerebral area, including cerebral blood flow (CBF) are affected by substance abuse and alcohol abuse, which causes brain atrophy in both male and female addicts resulting in increased lateral ventricles and larger volumes of cerebral spinal fluid (CSF).

U.S. patent application Ser. No. 13/061, 960 (U.S. Publication No. 20110208084) describes a method of detecting brain damage by measuring the bioimpedance of a brain region under investigation by use of at least one pair of current injecting electrodes and one pair of voltage sensing electrodes placed around the periphery of the brain region. This method describes how bioimpedance derived spectral information can be used to detect pathological changes in brain tissues such as brain bleeding, swelling or lesion growth. The method and apparatus described in the aforementioned patent application is however not sensitive to differentiating between the bioimpedance measurements resulting from the extra cranial scalp tissue and the intra cranial brain tissue and fluid. This results in a significant disadvantage of the inability to record accurate measurements of changes to the intra cranial brain tissue and fluid. The method and apparatus may be able to detect and monitor bleeding and cell swelling resulting from severe trauma but may not be sensitive to more subtle changes occurring from long term disorders such as substance abuse and other pathologies. US patent WO2002087410 A2 describes a method for diagnosis of mental disorders through analysis of an electric signal transmitted through the brain. This method like other prior art does not provide a way of removing signal originating from the scalp.

Therefore there is a need in the art to filter or reduce at least to some degree the impedance measurements resulting from extracranial tissue. This would result in a more sensitive determination of neural tissue impedance.

### SUMMARY OF THE PRESENT INVENTION

Disclosed is a method for determining brain state comprising the steps of a) attaching at least 2 first electrodes to the scalp of a subject at a separation; b) transmitting an electrical current between the at least 2 first electrodes wherein the current has one or more frequencies within a predetermined frequency spectrum; c) measuring a first impedance between the at least 2 first electrodes to provide a resulting impedance. Further described is the method having the further step of d) comparing the resulting impedance to pre-stored impedance measurements of the brain for normal and pathological state.

A further method is described for determining scalp impedance wherein the at least 2 first electrodes are positioned in a combined electrode unit having a center electrode and a peripheral electrode having a separation between wherein the separation is less than a maximum scalp thickness further comprising the step of: d) comparing the resulting impedance to pre-stored impedance measurements of the scalp for normal and pathological state. Also described is the method wherein the scalp impedance measurements are measured over a plurality of points on the scalp using more than one combined electrode units.

Further described is a method wherein the brain state can be more accurately determined by filtering out scalp impedance measurements wherein the separation between the at least 2 first electrodes is less than maximum scalp thickness, the method further comprising the steps of: d) attaching a reference electrode to the scalp of a subject wherein the separation between the reference electrode and at least one of the 2 first electrodes is at least maximum scalp thickness; e) transmitting a electrical current between the at least one of the 2 first electrodes and the reference electrode having a separation at least maximum scalp thickness wherein the current has one or more frequencies within a predetermined frequency spectrum; f) measuring a second impedance between the at least one of the 2 first electrodes and the reference electrode having a separation at least maximum scalp thickness; g) subtracting the first impedance from the second impedance to provide a resulting impedance; and h) comparing the resulting impedance to pre-stored impedance measurements for normal and pathological state.

Further described is the method for removing scalp impedance measurements wherein the at least 2 first electrodes are positioned in a combined electrode unit having a center electrode and a peripheral electrode having a separation between wherein the separation is less than a maximum scalp thickness and wherein the second impedance is measured between the center electrode of the combined electrode unit and the reference electrode.

Also described is the method further comprising the steps of a) continually evaluating bioimpedance measurements for existence and degree of abnormality; b) storing the information in the subject history; and c) treating the subject with a neuromodulatory technique. Additionally, the method is described wherein the at least 2 first electrodes are positioned in a combined electrode unit having a center electrode and a peripheral electrode having a separation between wherein the separation is less than a maximum scalp thickness method further comprising d) attaching at least a reference electrode to the scalp of a subject; e) attaching at least a first voltage electrode and second voltage electrode to the scalp of the subject where the separation between the voltage electrodes and the reference electrode is at least the maximum scalp thickness f) transmitting a predetermined electrical current between one first electrode comprised of a center electrode of the combined electrode unit and the third electrode wherein the current has one or more frequencies within a predetermined frequency spectrum; g) measuring a second impedance between the first voltage electrode and the second voltage electrodes; h) subtracting the first impedance from the second impedance to provide a resulting impedance; and i) comparing the resulting impedance to pre-stored impedance measurements for normal and pathological state.

Also described is the method wherein the at least 2 first electrodes are positioned in a combined electrode unit having a center electrode and a peripheral electrode having a separation between wherein the separation is less than a maximum scalp thickness method further comprising d) attaching at least a current electrode to the scalp of a subject; e) attaching at least a reference electrode to the scalp of a subject; f) attaching at least a first voltage electrode and second voltage electrode to the scalp of the subject where the separation between the voltage electrodes and the at least a current electrode is at least the maximum scalp thickness; g) transmitting a predetermined electrical current between the current electrode and the reference electrode wherein the current has one or more frequencies within a predetermined frequency spectrum; h) measuring a second impedance between the first voltage electrode and the second voltage electrodes; i) subtracting the first impedance from the second impedance to provide a resulting impedance; and j) comparing the resulting impedance to pre-stored impedance measurements for normal and pathological state.

Also described is a method for determining brain state comprising the steps of a) attaching at least 2 first electrodes to the scalp of a subject at a separation; b) attaching at least 2 second electrodes to the scalp of the subject; c) transmitting a predetermined electrical current between the at least 2 first electrodes wherein the current has one or more frequencies within a predetermined frequency spectrum; d) measuring a first impedance between the at least 2 second electrodes to provide a resulting impedance.

Further described is the method wherein the at least 2 first electrodes are comprised of a current electrode and a reference electrode, wherein the separation between the at least 2 first electrodes is at least maximum scalp thickness and wherein the 2 second electrodes consist of the current electrode and the center electrode of a combined electrode unit wherein a potential measured at the center electrode is forced onto the peripheral electrode by an operational amplifier, method further comprising: d) comparing the resulting impedance measurements to pre-stored impedance measurements for normal and pathological state.

Further described is the method wherein the at least 2 first electrodes are comprised of a center electrode of a driven shield combined electrode unit and a reference electrode, wherein the predetermined distance between the at least 2 first electrodes is at least maximum scalp thickness and wherein the at least 2 second electrodes are comprised of 2 center electrodes of 2 driven shield combined electrode units method further comprising the step of: d) comparing the resulting impedance measurements to pre-stored impedance measurements for normal and pathological state.

Additionally described is the method wherein the at least 2 first electrodes are comprised of a current electrode and a reference electrode, wherein the predetermined distance between the 2 first electrodes is at least maximum scalp thickness and wherein the at least 2 second electrodes are comprised of 2 center electrodes of 2 driven shield combined electrode unit method further comprising the step of: d) comparing the resulting impedance measurements to pre-stored impedance measurements for normal and pathological state.

Further described is the method wherein the at least 2 first electrodes are comprised of a center electrode of a driven shield combined electrode unit and a reference electrode, wherein the predetermined distance between the at least 2 first electrodes is at least maximum scalp thickness and wherein the at least 2 second electrodes are comprised of 2 center electrodes of 2 driven shield combined electrode units method further comprising the step of: d) comparing the resulting impedance measurements to pre-stored impedance measurements for normal and pathological state.

Also described is the method further comprising the steps of a) continually evaluating bioimpedance measurements for existence and degree of abnormality; b) storing the information in the subject history; and c) treating the subject with a neuromodulatory technique.

Alternatively, the method is described wherein the at least 2 first electrodes are comprised of a center and peripheral electrode of a combined electrode unit wherein the predetermined distance between the at least 2 first electrodes is at least maximum scalp thickness method further comprising the steps of d) attaching at least 1 driven shield combined electrode unit to the scalp of the subject; e) transmitting an electrical current between the center electrode of at least 1 combined electrode unit and the reference electrode, wherein the current has one or more frequencies within a predetermined frequency spectrum; f) measuring a second impedance between 2 center electrodes comprised of 1 center electrode of the combined electrode unit and 1 center electrode of the driven shield combined electrode unit; g) subtracting the first impedance from the second impedance to provide a resulting impedance measurement; and h) comparing the resulting impedance measurements to pre-stored impedance measurements for normal and pathological state.

Further described is the method wherein the at least 2 first electrodes are comprised of a center and peripheral electrode of a combined electrode unit wherein the predetermined distance between the at least 2 first electrodes is at least maximum scalp thickness method further comprising the steps of d) attaching at least 2 driven shield combined electrode units to the scalp of the subject; e) attaching at least 1 reference electrode to the scalp of the subject; f) transmitting a predetermined electrical current between the center electrode of at least 1 combined electrode unit and 1 reference electrode wherein the current has one or more frequencies within a predetermined frequency spectrum; g) measuring the impedance between the center electrodes of the at least 2 driven shield combined electrode units; h) subtracting the first impedance from the second impedance to provide a resulting impedance measurement; and i) comparing the resulting impedance measurements to pre-stored impedance measurements for normal and pathological state.

Also described is the method wherein the at least 2 first electrodes are comprised of a center and peripheral electrode of a combined electrode unit wherein the predetermined distance between the at least 2 first electrodes is at least maximum scalp thickness method further comprising the steps of: d) attaching at least 3 driven shield combined electrode units to the scalp of the subject; e) attaching at least 1 reference electrode to the scalp of the subject; f) transmitting a predetermined electrical current between the center electrode of at least 1 driven shield combined electrode unit and 1 reference electrode wherein the current has one or more frequencies within a predetermined frequency spectrum; g) measuring the impedance between the center electrodes of the at least 2 driven shield combined electrode units; h) subtracting the first impedance from the second impedance to provide a resulting impedance measurement; and i) comparing the resulting impedance measurements to pre-stored impedance measurements for normal and pathological state.

Described herein is a combined electrode unit for determining scalp state comprising an inner electrode having a central conductive area, a surrounding non-conductive insulating area, and an outer electrode consisting of a peripheral conductive area, wherein a separation between an edge of the central conductive area and the peripheral conductive area is less than maximum scalp thickness. An embodiment wherein the voltage measured at the inner electrode is forced on the peripheral conductive area by an operational amplifier is also described.

### BRIEF DESCRIPTION OF THE DRAWINGS

The method and apparatus will be hereinafter described with references to the accompanying drawing and diagrams. The following descriptions can be considered as a preferred embodiment but not in the limiting sense.

FIG. 1 is a high-level functional diagram of the system, according to one embodiment of the present invention.

FIG. 2 is a view of the layout of the specialized spectrometer, according to one embodiment of the present invention.

FIG. 3 is a view of the specialized spectrometer connected to the patient, according to one embodiment of the present invention.

FIG. 4 is a diagram showing a preferred electrode montage, according to one embodiment of the present invention.

FIG. 5 shows circular and rectangular embodiments of combined electrode units, according to one embodiment of the present invention.

FIG. 6 is an illustration of 2 circular driven shield combined electrode units, according to one embodiment of the present invention.

FIG. 7 show a driven shield current detection unit placed on a resistor model, according to one embodiment of the present invention.

FIG. 8 shows the application of a 3 electrode configuration to a head, according to one embodiment of the present invention.

FIG. 9 shows the application of a 3 electrode configuration to a head, according to one embodiment of the present invention.

FIG. 10 shows the application of a combined electrode unit and a reference electrode to a head according to one embodiment of the present electrode.

FIG. 11 shows the application of a combined electrode unit, 2 voltage electrodes and a reference electrode to a head according to one embodiment of the present electrode.

FIG. 12 shows the application of a combined electrode unit, 1 current electrode, 2 voltage electrodes and a reference electrode to a head according to one embodiment of the present electrode.

FIG. 13 shows the application of a combined electrode unit to a head, according to one embodiment of the present invention.

FIG. 14 shows the application of a driven shield combined electrode unit, 1 current electrode and 1 reference electrode to a head, according to one embodiment of the present invention.

FIG. 15 shows the application of 2 driven shield combined electrode units, and 1 reference electrode to a head, according to one embodiment of the present invention.

FIG. 16 shows the application of 2 driven shield combined electrode units, 1 current electrode and 1 reference electrode to a head, according to one embodiment of the present invention.

FIG. 17 shows the application of 3 driven shield combined electrode units, and 1 reference electrode to a head, according to one embodiment of the present invention.

FIG. 18 shows the application of 1 combined electrode unit, 1 driven shield combined electrode units, and 1 reference electrode to a head, according to one embodiment of the present invention.

FIG. 19 shows the application of 1 combined electrode unit, 2 driven shield combined electrode units, and 1 reference electrode to a head, according to one embodiment of the present invention.

FIG. 20 shows the application of 1 combined electrode unit, 3 driven shield combined electrode units, and 1 reference electrode to a head, according to one embodiment of the present invention.

FIG. 21 shows the application of multiple combined electrode units at different locations on a scalp according to one embodiment of the present invention.

FIG. 22 is a flowchart showing the intracranial brain pathology assessment process, according to one embodiment of the present invention.

FIG. 23 is a flowchart showing the data processing and classification engineflow, according to one embodiment of the present invention.

FIG. 24 shows the a treatment loop flow of the classification engine, according to one embodiment of the present invention.

FIG. 25 shows an example of an output of the classifier, according to one embodiment of the present invention.

### DETAILED DESCRIPTION OF THE PRESENT INVENTION

It is an object of the present invention to provide a method and system that can measure, detect, monitor and analyze changes in intracranial tissue and fluid, composition and volume that are related to the state and progress of addiction including periods of active addiction and recovery periods of abstention. The method and system described can also be used to measure, detect and analyze changes in intracranial fluid and brain tissue and extracranial tissue related to other pathological states such as stroke, tumor, hematoma and hypoxia.

The basic method described in the patent consists of the steps of applying at least one pair of current electrodes on the scalp or outer skin layer around the periphery of the brain region undergoing analysis, generating an electric stimulus either in current or voltage form, measuring the resulting voltage drop between the pair and calculating the impedance of the brain tissue of interest. More specialized methods and apparatus described in the patent provide a method to remove the scalp impedance from total impedance measurements. The transmitted current may consist of transmitting a single frequency or a range of frequencies within a defined spectrum, depending on the mode of application and the settings desired by the operator. Various analytics and plots may be derived from the calculated impedance including but not limited to, total brain fluid volume, resistance and reactance calculated for every frequency, Bode plots of impedance magnitude vs. frequency, Cole-Cole plots and plots of resistance-reactance spectrums for every frequency. All the derived analytical measures including numerical values and plots may be compared with data from non-addicted or healthy individuals for detection of any abnormalities related to the state and progress of the addiction or other pathologies and disorders. This comparison may be done exclusively by a trained operator, e.g. addiction specialist, for example by visual comparison of normal vs. addict bioimpedance derived spectral plots or numerical values. Alternatively classification engines designed from software based learning models such as artificial neural networks or support vector machines will be trained to recognize and identify abnormalities in bioimpedance data and its derived analytics measured from addict brains. The classification engine will be designed to complement the expertise of the operator who will analyze the data. A database of bioimpedance measurements of both healthy and addicted individuals may be used to assist the classification of the measured bioimpedance signal. Furthermore the classification engine may provide useful analytics such as addiction type, severity index of addiction and history of brain composition changes over a series of measurements. Brain composition changes and/or brain changes related abstention from drugs of abuse may be tracked over a period of addiction treatments such as neuromodulation techniques including Transcranial Direct Current Stimulation (tDCS).

The preferable embodiment of the invention will be non-invasive, portable and mobile allowing usage of the device both in and out of a hospital setting.

The electrical stimulus may be either from a current or voltage source as long as the stimulation results in injection of electrical current between the external electrodes and through the tissues under analysis.

The voltage drop between the electrode pair may be measured on a single measurement channel or on multiple measurement channels.

The transmitted current may be DC, a single frequency, multiple frequencies or a frequency sweep within a known frequency range. In addition the transmitted current may contain multiple frequencies such as step functions or white noise waveforms.

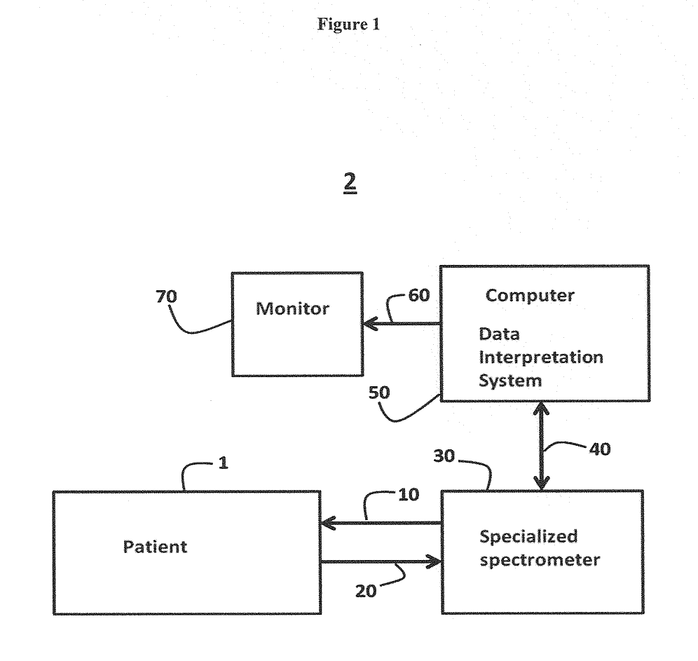

FIG. 1 illustrates the layout of the major components and process of the measurement and monitoring system 2. The specialized spectrometer 30 is connected to the patient by way of lines 10 and 20. Lines 10 and 20 symbolize all information or measurements that are either outputted 10, by the spectrometer to the patient 1, or are inputted 20, from the patient to the specialized spectrometer. The specialized spectrometer is connected through communication link 40 to a data interpretation system 50, for example a PC or another form of computer or central processing unit having interpretation software therein. The flow of information through 40 can pass back and forth between the specialized spectrometer to the data interpretation system as indicated by the double arrows. The data interpretation system can output data through communication link 60 to a monitor 70 or another form of display, which displays information for the user.

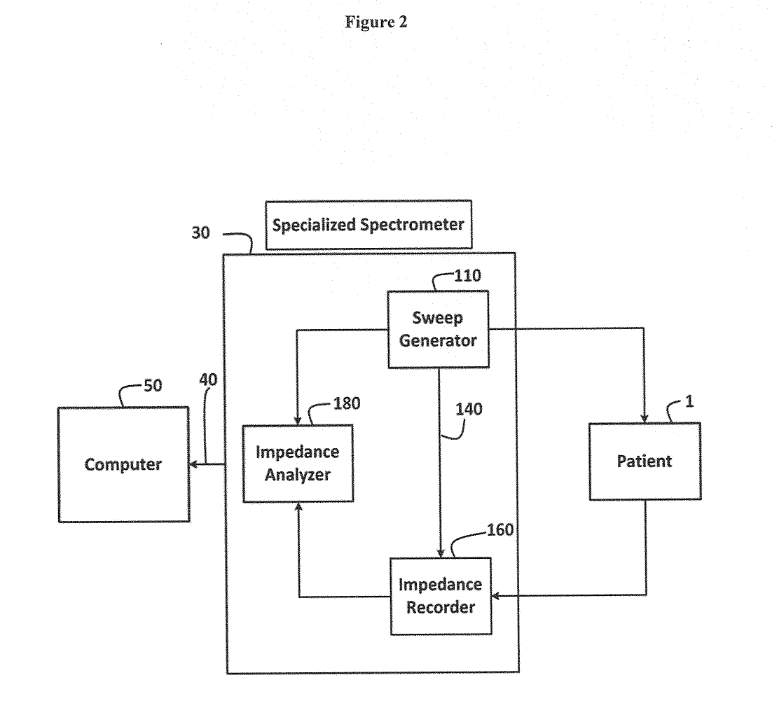

FIG. 2 shows a more detailed view of the specialized spectrometer 30 and its major components, composed of a frequency sweep generator 110, an impedance recorder 160, and an impedance analyzer 180. The frequency sweep generator generates an electrical current at set frequencies within a defined frequency band that is applied to the patient 1. A resulting alternating voltage is recorded in the impedance recorder 160, which digitally records the data using non-volatile memory. The data is further transmitted and stored in the impedance analyzer 180, where processing occurs that allows transmission of the data to a data interpretation system generally a computer 50, through communication link 40, for the purpose of further signal processing and analysis and visual display of the data. The sweep generator operation of applying electrical current to the patient is synchronized to the input of the impedance recorder as well as the impedance analyzer as shown by the solid arrows projecting out of the sweep generator.

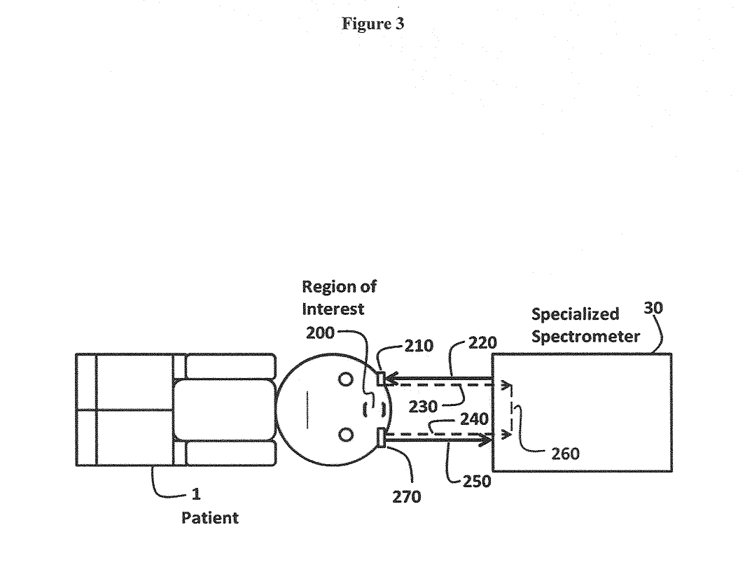

FIG. 3 illustrates a two-electrode configuration embodiment of the bioimpedance measurement system. The specialized spectrometer 30 is connected through lead wire 220 to a cutaneous current electrode 210 on the outer scalp tissue of the patient 1. The current is transmitted through the electrode—tissue interface and through the principal tissue layers including the scalp, skull, cerebrospinal fluid and neural tissues. Electrodes 210, 270 are situated around the periphery of the region of interest 200 so that at least a portion of the current passes through the intracranial region of interest 200. The current is received by electrode 270 and follows the current return path through lead wire 250 into the specialized spectrometer 30. Electric potential measurements are taken at the sites of current injection and reception electrodes 210 and 270 respectively, through wires 230 and 240. The voltage drop between the two measurement points indicated by 260 can be measured, thus providing information on the bioimpedance of the intracranial region of interest. Analysis of the bioimpedance measurements including phase and amplitude of the real and imaginary components can be used to discern and monitor potential pathological states in the region of interest.

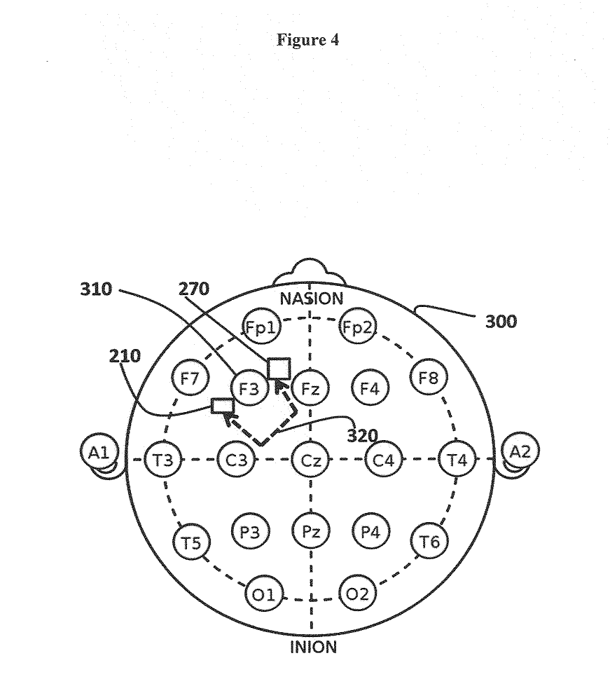

FIG. 4 shows preferred locations for the two electrode configuration on the head of a subject. A standard 10-20 EEG graph of electrode sites 300 shows electrodes 270 and 210 situated around the periphery of the region of interest, the F3 site 310, otherwise known as the left dorsolateral prefrontal cortex (DLPFC). The current propagation path includes passage via the F3 site. The voltage drop measurement 320 measured across F3 provides bioimpedance data of the region of interest. Since changes in grey matter density at the left DLPFC have been observed in certain categories of addicts, knowledge of the bioimpedance data of this intracranial region will assist in the assessment and monitoring of the addiction state. Other montages may be utilized to investigate various regions of interests depending on the nature of the pathological state being investigated.

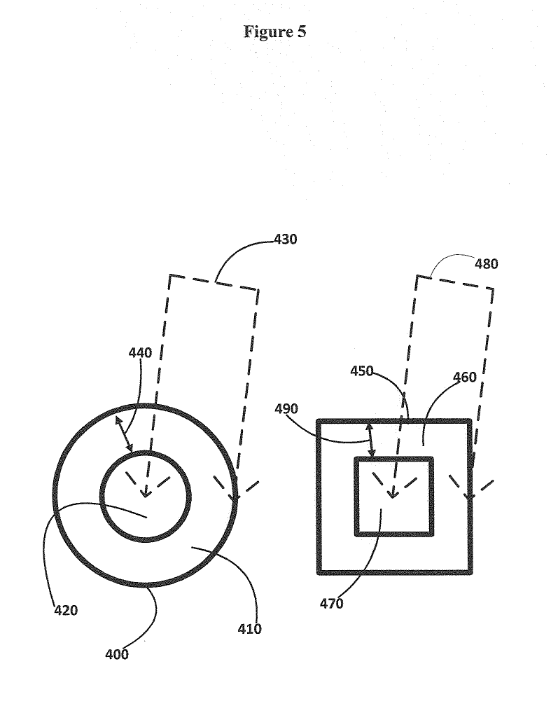

FIG. 5 shows a plan view of a combined electrode unit illustrated in round and square geometry. The electrodes may be used to transmit and/or receive current, to detect voltage potential, or both. The major components of the electrode shown for round geometry are an inner electrode comprised of a conductive core 420, a surrounding non conductive area 410, and an outer electrode comprised of a peripheral conductive area 400. The major components of the combined electrode unit shown for square geometry are an inner electrode comprised of conductive area 470, a surrounding non conductive area 460, and an outer electrode comprised of peripheral square conductive layer 450. The combined electrode units may be scaled to any desired dimension and designed in any geometrical shape as long as the distance or separation between the outer edge of the inner conductive area and the surrounding peripheral conductive area is less than the maximum scalp thickness of the region where the electrodes are applied. The scalp thickness may vary at different locations and inter-individually. Some examples for scalp thickness reported from the literature are an average value of scalp thickness of 9.5±2.6 mm in the temporal area, 6.6±1.3 mm in the parieto-occipital area and 4.8±1.1 mm in the frontal area. Thus the gap between the center and peripheral electrodes of the combined electrode unit should depend on the subject specific scalp thickness as well as the electrode montage that will be used. This distance is symbolized by arrows 440 and 490 and for most applications will be assumed to have a gap under 5 mm. As an example throughout, the 5 mm distance will be referred to from now on as the “maximum scalp thickness”, however the maximum scalp thickness depends on the thickness of the patient's scalp and may range from less than 1 mm to greater than 12 mm. Electric potential measurements can be taken at the inner and outer conductive regions respectively as shown by the position of the dashed arrows. Differences between the potential measurements (commonly known as the voltage drop) may be measured between the inner conductive area 420 and outer peripheral conductive area 400, for the round geometry embodiment as shown by 430, and between the inner conductive area 470 and outer peripheral conductive area 450, for the square geometry embodiment as shown by 480. The voltage drop can measured between the inner and outer conductive regions over a transmitted current within a known frequency range. Since the extracranial scalp tissue is assumed to be not more than 5 mm thick, impedance measurements between the inner and outer electrodes of the combined electrode units will relate entirely or largely to the extracranial scalp tissue impedance between the voltage measurement points. Thus applying a current over a known frequency range to the subject through an inner electrode of the combined electrode unit, receiving the current at the peripheral electrode, measuring the voltage drop between the inner and outer electrodes, and calculating the resulting impedance over the frequency range, provides a method to measure the impedance of tissue in the extracranial region separately from the impedance of the skull and intracranial tissue. This bioimpedance data or signal may be filtered from the total impedance data that includes impedance of the scalp, skull and brain, to provide a more accurate method of measuring the impedance of the intracranial tissue.

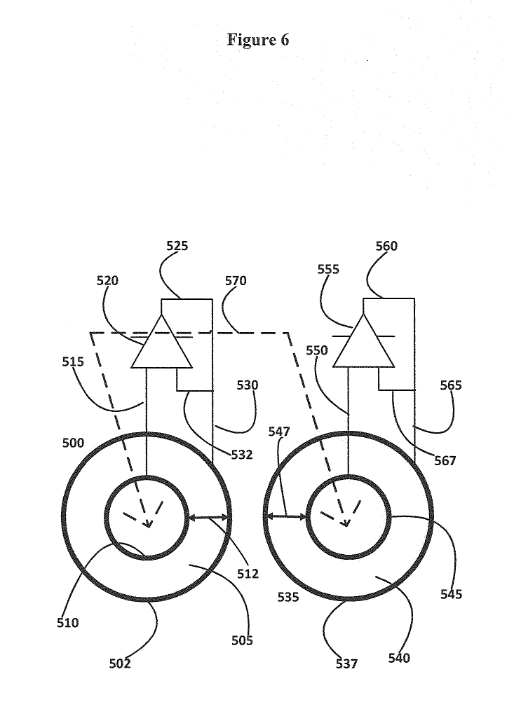

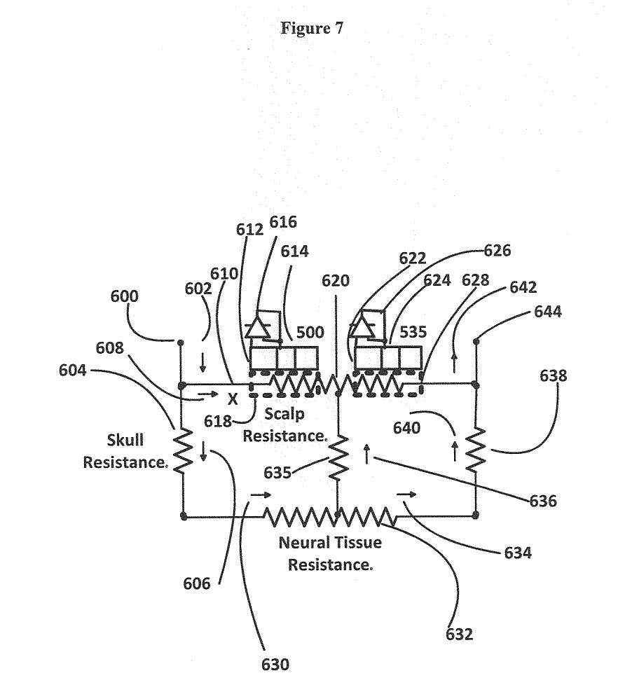

FIG. 6 illustrates a plan view of an arrangement of two combined electrode units of circular design 500 and 535, as described in FIG. 5 comprised of an inner conductive core 510 and 545, a surrounding non conductive layer 505 and 540, and a peripheral conductive layer 502 and 537. The electrode on the left of FIG. 6 is connected between the inner conductive core 510 and a operational amplifier commonly referred to as a voltage buffer 520, through input wire 515. The output wire 525 acts as a feedback and connects to input 532. The voltage buffer 520 forces input 532 to assume the same potential as input wire 515, which in turn forces the potential value on the wire 530 and onto the peripheral conductive layer 502. Thus the effect of the voltage buffer is to force the peripheral conductive layer to assume the same potential as measured at the inner conductive layer. This design is commonly known as a driven shield electrode. A similar configuration of the combined electrode unit connected to a voltage buffer is shown for the electrode on the right of FIG. 7 where the inner conductive layer 545 is connected to the voltage buffer 555 through input 550. The output 560 feeds back to input 567. The voltage buffer 555 forces input 567 to assume the same voltage potential as input 550, which in turn forces the potential value on the wire 565 and onto the peripheral conductive layer 537. The high impedance of the voltage buffer will prevent significant amounts of current from flowing through the amplifier from one conductive region to the other. The voltage drop 570 measured between the conductive cores 510 and 545 for each frequency of the transmitted current can be converted into impedance measurements. These impedance measurements will not include at least in large part, the impedance of the extracranial scalp due to the forced isopotential between the conductive core and peripheral conductive layer which prevents current from propagating totally or in part tangentially along the scalp from the conductive core to the peripheral conductive layer. Thus this describes a method that improves accuracy of measurement of the impedance of the intracranial tissue.

FIG. 7 illustrates in greater detail the principle of the driven shield electrode design. A network of resistors in parallel and in series, are used to model to resistance of the scalp tissues 620, skull resistance 604, 635 and 638, and resistance of the neural tissue 632. The capacitive element of the tissues is not modeled. A split view of the coronal plane of the driven shield electrodes 500 and 535 is shown. A current electrode is used to inject current into the scalp through input 600. An electrode receives the transmitted current at point 644 as shown by the flow of current at 642. The direction of the injected current is shown by the arrow 602. When the current reaches the nodal point, represented by inner scalp tissue, part of current propagates tangentially to the outer scalp layer as shown by arrow 608 and part of the current propagates radially through the skull as shown by arrow 606. Guarded shield electrodes 500 and 535 can be placed on the scalp and the voltage drop caused by the current flow introduced at 600 can be measured between their respective conductive cores. The potential sensed at the conductive cores 614 and 624 of the guarded shield electrodes are forced onto the peripheral conductive layers 612 and 622, by way of voltage buffers 616 and 626 respectively. The flow of current tangential to the outer scalp 608, can be restricted entirely or partly as symbolized by the uppercase X 610 due to the isopotential forced between the inner 614 and peripheral 612 conductive core as shown by the dashed rectangular area 618. In a similar fashion the flow of the current 636 travelling radially through the skull resistance 635 towards the scalp resistance 620 can be restricted entirely or partly from flowing through scalp resistance 620 towards output 644 by forcing an isopotential area 628. Thus the contribution of the scalp resistance to the voltage drop measured between 614 and 624 is partly or entirely removed. The voltage drop measured between the electrodes 500 and 535 is therefore largely comprised of the resistance encountered by current 606, and 640 flowing through skull resistance 604, and 638 and the resistance encountered by current 630 and 634, flowing through neural tissue resistance 632. Thus the guarded shield electrodes provide a way to filter the impedance measurements of the extra-cranial scalp tissues.

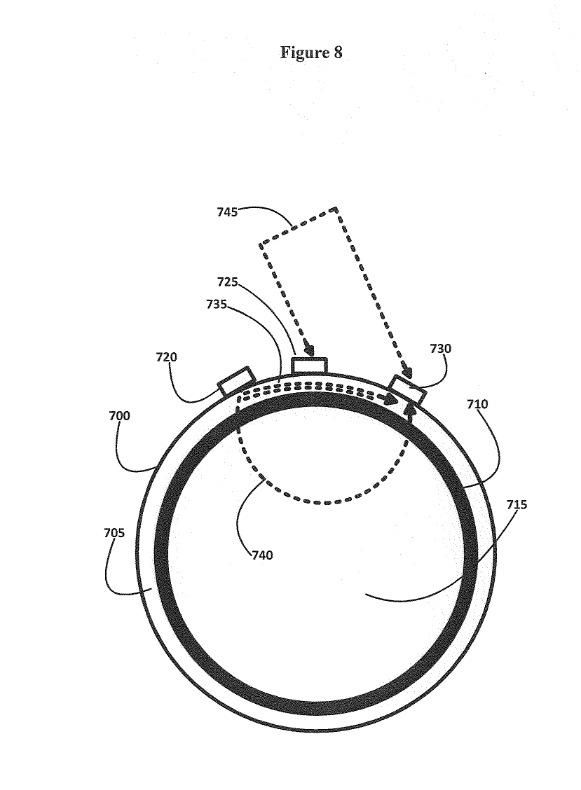

FIG. 8 illustrates one embodiment of a method of measuring bioimpedance of the intracranial tissue shown on a simplified head model consisting of concentric circles representing an outer scalp layer 700, scalp tissue 705, skull 710, and intracranial tissue and fluid 715. A split view of the coronal plane of the electrodes and the head model are shown. Current electrode 720, voltage electrode 725, and reference electrode for current reception 730, are all placed on the outer scalp 700. Current comprised of a single or multiple frequencies within a known frequency range is transmitted from electrode 720 to electrode 730. Electric potential can be measured at electrodes 725 and 730 in order to measure the voltage drop between these points. Alternatively a voltage drop can be measured between electrodes 720 and 725. The three electrode configuration is advantageous since it partly eliminates the measurement of the high impedance layer of the outer skin. Current flow 735 through the conductive scalp is greater than the current flow through the highly resistive skull into the brain 740, as shown by the double dotted lines of current flow 735. Thus the total measured impedance at low frequencies may be mainly comprised of the impedance of the extracranial scalp tissue in addition to the impedance of the skull and neural tissue.

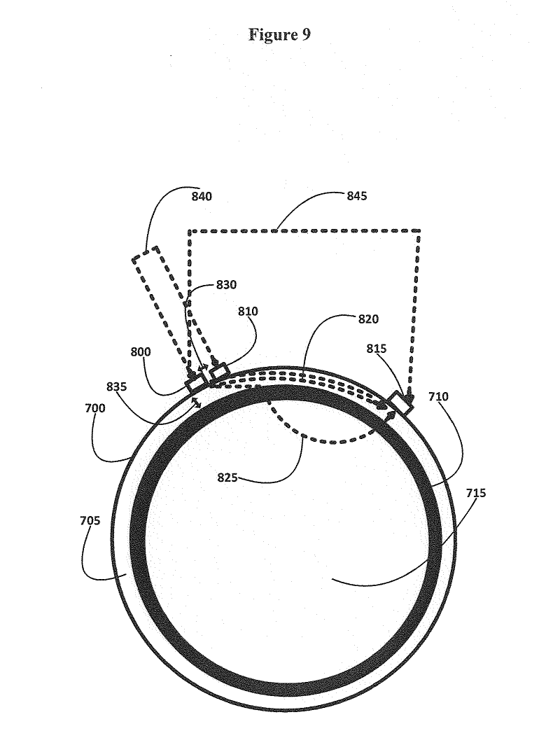

FIG. 9 illustrates an embodiment of a method that may be used to at least partly remove the impedance of the extracranial scalp from the total impedance measurements allowing for a more accurate determination of the impedance of the neural tissue. Current electrode 800, voltage electrode 810 and reference electrode 815 are shown placed on the scalp layer 700 of the simplified head model described in FIG. 8. The distance between electrodes 800 and 810, symbolized by the double arrow 830, is set to less than the maximum scalp thickness shown by double arrow 835. Current comprised of a single or multiple frequencies within a known frequency range is transmitted from electrode 800 to electrode 815. Since the distance between these electrodes is less than the maximum scalp thickness 835, the potential drop measured between 800 and 810 is caused largely or entirely of current flow through the scalp tissue as shown by the segments of current 820 and 825 normal to electrode 810. Thus the impedance measured between 800 and 810 is largely or entirely the scalp tissue impedance. Another voltage drop can be measured between electrodes 800 and 815 where a greater distance than the maximum scalp thickness separates them. This voltage drop can be used to calculate the impedance resulting from current passage through the scalp, skull and neural tissue as shown by the passage of current 820 and 825. The impedance of the scalp measured between 800 and 810 can be subtracted from the impedance of the scalp, skull and neural tissue measured between 800 and 815. This provides a method of removing the scalp impedance from the total measured impedance.

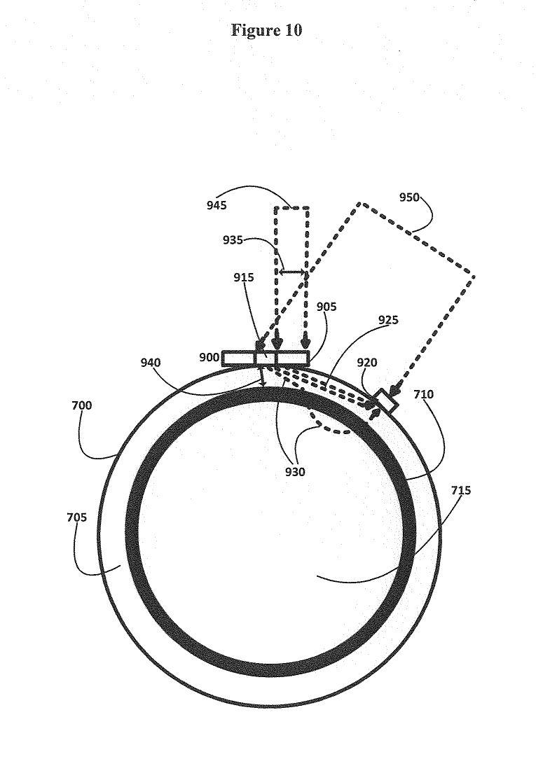

FIG. 10 illustrates another embodiment of the method described in FIG. 9. A combined electrode unit 900, as described above with reference to FIG. 5, and a reference electrode 920, are placed on the scalp layer 700. The distance between the inner 915 and outer 905 electrodes of the combined electrode unit is less than the maximum scalp thickness 940 represented by the double arrow 935 thus the voltage drop of current 930 and 925 measured between the inner electrode 915 and outer electrode 905 as shown by 945, is used to calculate largely or entirely the scalp tissue impedance. Another voltage drop can be measured between the center electrode 915 of the combined electrode unit 900 and reference electrode 920. Since the distance between electrodes 915 and 920 is greater than the maximum scalp thickness, the voltage drop is caused by current flow 925 through the scalp and current flow 930, through the scalp, skull, and neural tissue. Scalp impedance measured between 915 and 905 can be subtracted from the total impedance measured between 915 and 920 to determine intercranial impedance.

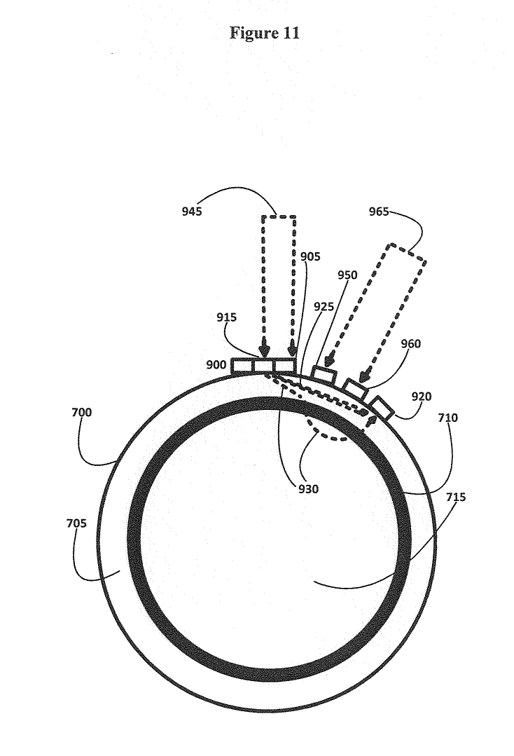

FIG. 11 is a variation of the embodiment described in FIG. 10. A combined electrode unit 900, voltage electrode 950, voltage electrode 960 and reference electrode 920 are placed on the scalp layer 700. Current is transmitted between inner electrode 915 and reference electrode 920. Scalp impedance caused by current flow 925 and 930 can be calculated by measuring the voltage drop 945, between 915 and 905. Total impedance of the scalp, skull and neural tissue can be calculated by measuring the voltage drop 965 of current 925 and 930 between electrodes 950 and 960. Separating voltage measurements from current sources provides the benefit of removing impedance at the electrode-tissue interface from the impedance measurements. Scalp impedance measurements can then be subtracted from the total impedance.

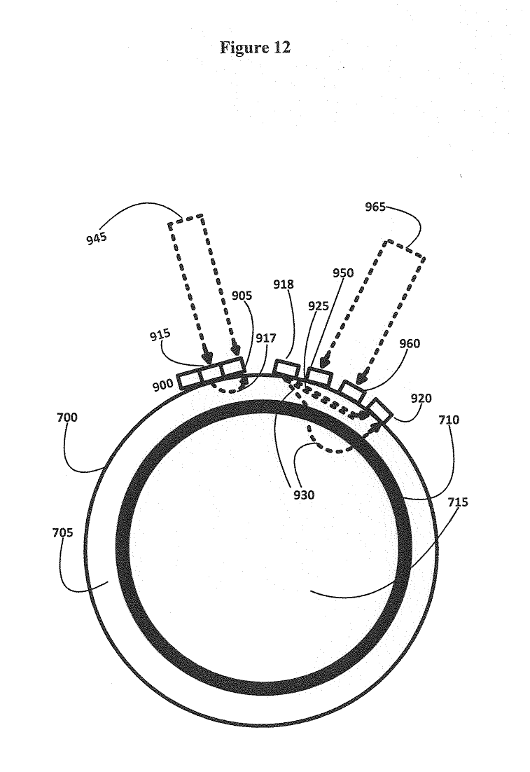

Another embodiment of the method illustrated in FIG. 11 is shown in FIG. 12. In addition to combined electrode 900, reference electrode 920, and voltage electrodes 950 and 960, an additional current electrode 918 is placed on the scalp layer. Current is transmitted independently between 915 and 905 of the combined electrode and between current electrode 918 and reference electrode 920. Measurement of the voltage drop 945 between inner electrode 915 and outer electrode 905 of the combined electrode unit can be used to calculate the scalp impedance. Measurement of voltage drop 965 can be used to calculate the total impedance of the scalp, skull and neural tissue. Scalp impedance measurements can then be subtracted from the total impedance measurements to provide a more accurate measurement of the intracranial impedance.

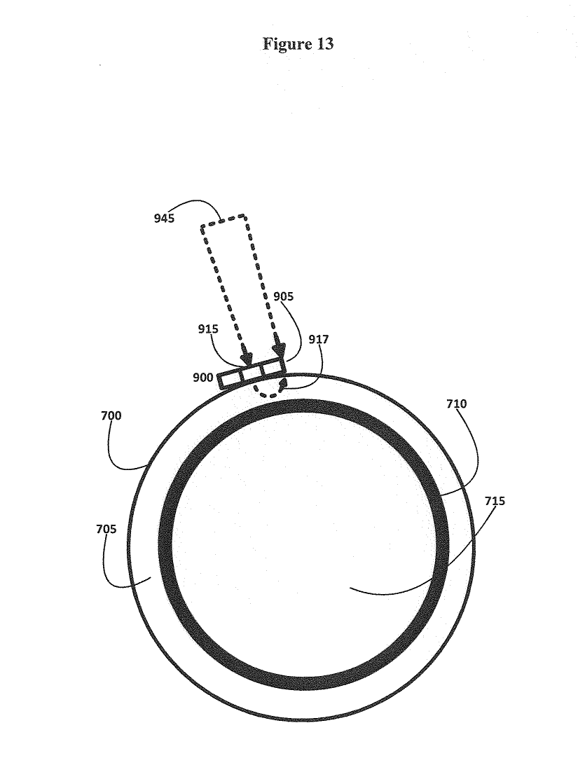

In some instances it use useful to know the impedance of the scalp separately from the impedance of other tissues for instance for pathologies that are only affecting the scalp. FIG. 13 shows that combined electrode unit 900 can be attached alone to the scalp of a subject. An electrical current can be transmitted between inner electrode 915 and outer electrode 905. The voltage drop 945 between these points can be used to calculate the scalp impedance.

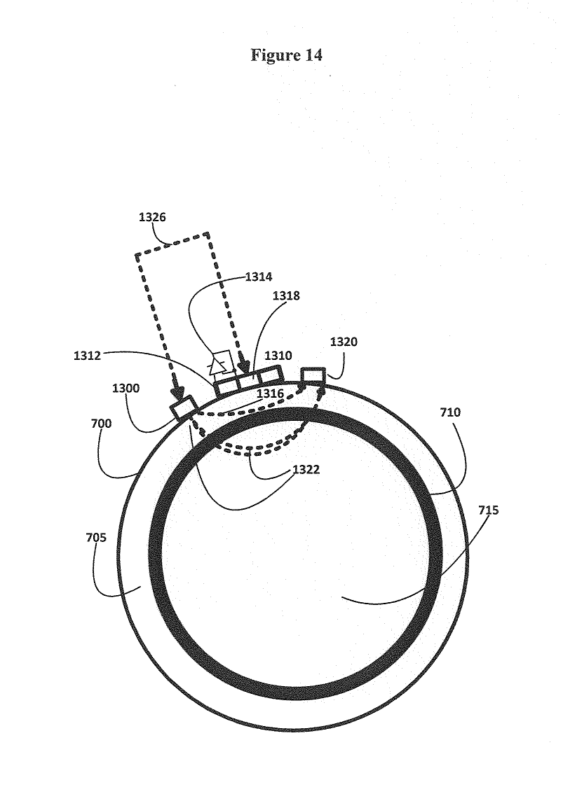

FIG. 14 illustrates another method that can be used to remove measurements of the scalp impedance from the total impedance. A combined electrode unit of driven shield design 1310, as described above with reference to FIG. 6, current electrode 1300, and reference electrode 1320 are shown attached to the scalp of the subject. Current is transmitted from electrode 1300 to electrode 1320 as shown by the path of the dotted lines 1316 and 1322. Since an isopotential area is generated between inner electrode 1318 and outer electrode 1312 through voltage buffer 1314, current propagation through the scalp is restricted. This is shown symbolically by illustrating that the current flow through the scalp tissue 1316 is comprised of a single dotted line showing limited flow, whereas current flow through the scalp, skull and neural tissue 1322 is comprised of double dotted lines. Thus the impedance calculated from the voltage drop 1326, measured between electrodes 1300 and 1318 is largely caused by current propagation through the skull and neural tissue rather than through the scalp. Another possible embodiment of the same method would be obtained by measuring the voltage drop between electrode 1318 and the reference electrode 1320.

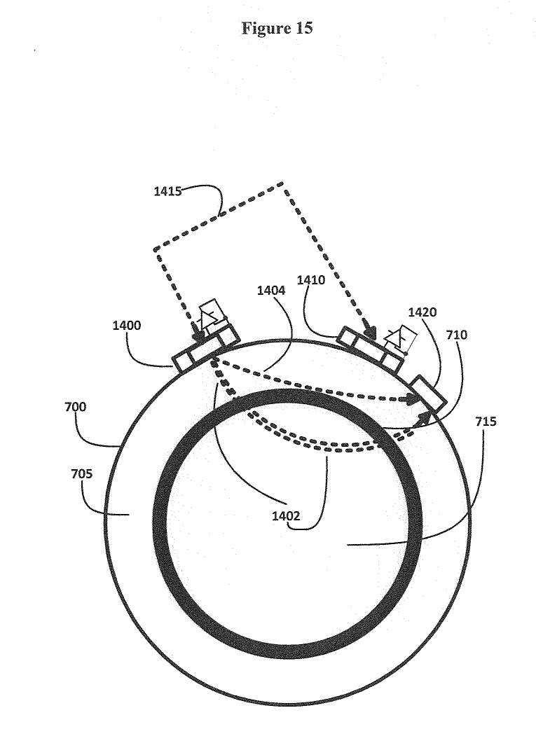

FIG. 15 demonstrates a further embodiment of the method described in FIG. 14. Driven shield combined electrode units 1400 and 1410 and reference electrode 1420 are all placed on the scalp of a subject. The isopotential generated between the inner and outer electrode of the driven shield combined electrode unit can be used to focus current propagation normal to the orientation of the scalp. This provides the benefit of adding further control of the current path and restricting current flow tangential to the scalp. Following this principal, current is transmitted between the inner electrode of the driven shield combined electrode unit 1400 and the reference electrode 1420. Current flow through the scalp represented by 1404, is restricted and is the non primary path as shown by single dotted line, whereas current flow through the scalp, skull and neural tissue 1402 is the primary path of current propagation as shown by the double dotted line. Thus the impedance calculated from the voltage drop measured between 1400 and 1420 is primarily caused by current propagation through the skull and neural tissue rather than the scalp.

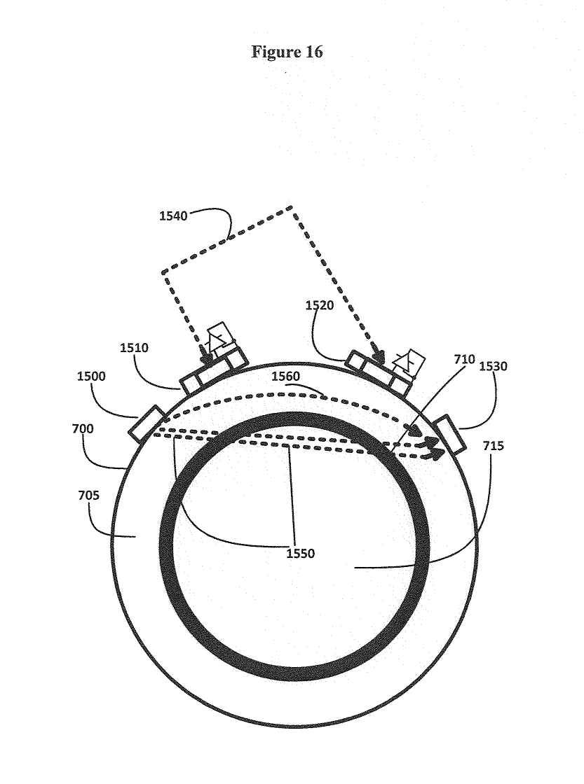

FIG. 16 shows another embodiment of the method described in FIG. 15. Current electrode 1500, driven shield combined electrode units 1510 and 1520, and reference electrode 1530 are placed on the scalp of a subject. Current is transmitted between 1500 and 1530 and the voltage drop 1540 is measured through driven shield combined electrode units 1510 and 1520. Separate electrodes for current transmission and reception and for voltage detection allow for removal of the measurement of impedance at the electrode tissue interface. The impedance calculated from the voltage drop 1540 is primarily impedance of the skull and neural tissue as shown by the primary current propagation path 1550 symbolized by the double dotted line as compared to the current path through the scalp 1560 symbolized by the single dotted line.

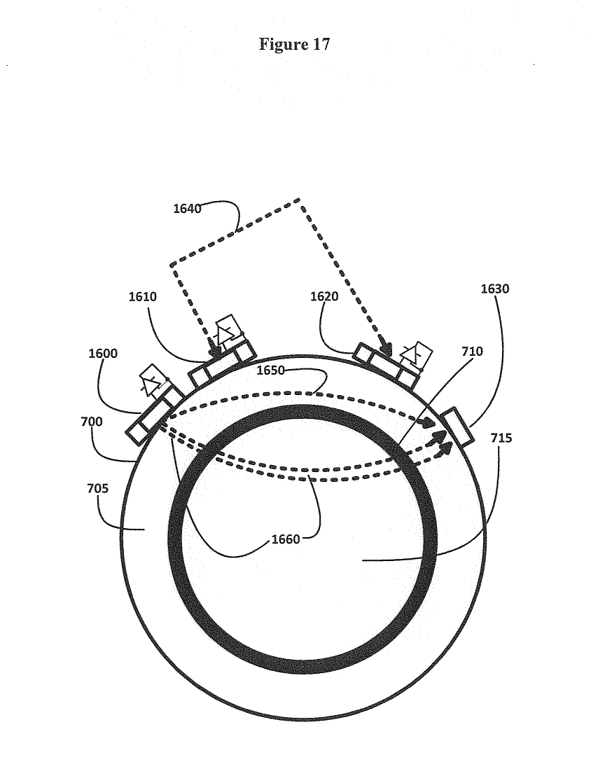

FIG. 17 is another embodiment of the method described in FIG. 16. In addition to the advantages of having separate current and voltage electrodes, a driven shield combined electrode unit is used to focus current delivery and to prevent current propagation through the scalp. In this embodiment, driven shield combined electrode units 1600, 1610, and 1620 and reference electrode 1630 are placed on the scalp of a subject. Current is transmitted from 1600 to 1630 and the voltage drop 1640 is measured between 1610 and 1620. Since the primary current path 1660 is through the scalp, skull and neural tissue and not through the scalp alone as shown at 1650, the impedance of the scalp is largely removed from the impedance measurements.

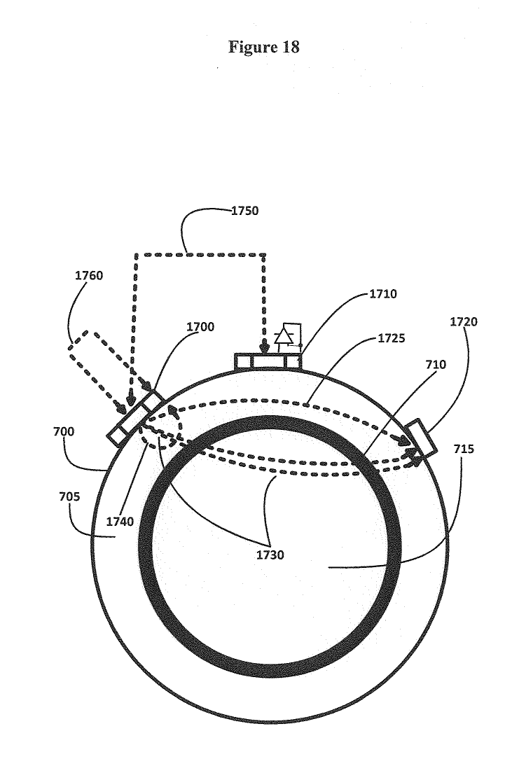

FIG. 18 describes a method of removing the scalp impedance measurement by combining the methods described for the combined electrode units described in FIG. 10-13 and the methods described for the driven shield combined electrode units described in FIG. 14-17. In this embodiment combined electrode unit 1700, driven shield combined electrode unit 1710 and reference electrode 1720 are attached to the scalp of a subject. Current is transmitted between the inner and outer electrode of the combined electrode unit 1700 as shown by current path 1740. The impedance calculated from the voltage drop 1760 is a result of current path through the scalp, thus only the scalp impedance is measured. In addition current is transmitted between the inner electrode of the combined electrode unit 1700 and reference electrode 1700. Current propagates through the scalp tissue as shown by current path 1725 and through the scalp, skull, and neural tissue as shown by current path 1720. A voltage drop is measured between the center electrodes of the combined electrode unit 1700 and the driven shield combined electrode unit 1710. Due to the isopotential region forced between the inner and outer electrodes of the driven shield combined electrode unit, the primary current impedance sensed through the voltage drop propagates through the scalp, skull and neural tissue as shown by the double line of current path 1730 as compared to the single line representing the current path 1725 solely through the scalp tissue. The scalp impedance measurements collected from the combined electrode unit can then be subtracted from the impedance measurements obtained from the combined electrode unit and the driven shield combined electrode unit. This provides a method of removing the scalp impedance from the total impedance measurements.

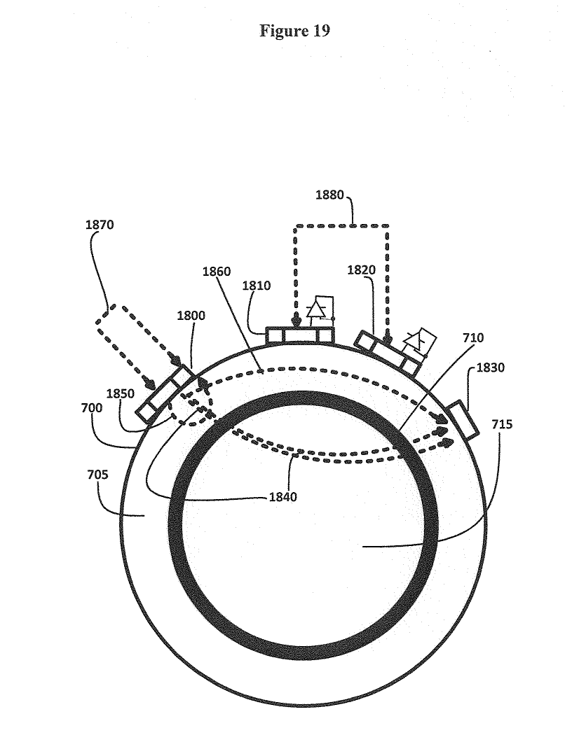

FIG. 19 demonstrates a variation of the embodiment described in FIG. 18. In this case, combined electrode unit 1800, driven shield combined electrode units 1810 and 1820, and reference electrode 1830 are placed on the scalp of a subject. The scalp impedance is measured by transmitting current from the inner electrode to the peripheral electrode of the combined electrode unit 1800 as shown by current path 1850, and measuring the resulting voltage drop as shown at 1870. Current is also transmitted from the inner electrode of the combined electrode unit 1800 to the reference electrode 1830. The voltage drop may be measured between the inner electrodes of the driven shield combined electrode units 1810 and 1820. The resulting impedance is mainly the result of the primary current path 1840 through the scalp, skull and neural tissue due to the isopotential areas that restrict current flow through the scalp as shown by current path 1860. The advantage of this configuration is that the measurements of the electrode tissue interface are eliminated due to the separation of voltage measurements and current transmission. The scalp impedance measurements collected from the combined electrode unit can then be subtracted from the impedance measurements obtained from the driven shield combined electrode units.

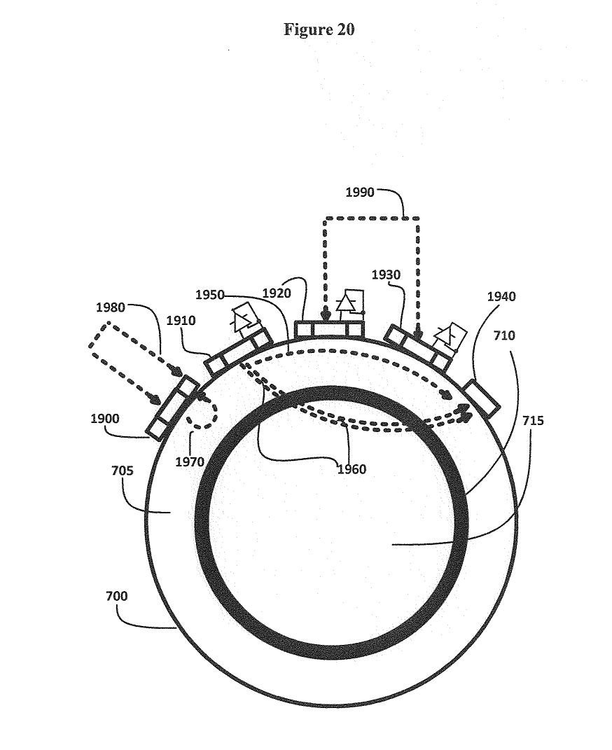

FIG. 20 is a further embodiment of the method presented in FIG. 19. In addition to separate voltage measurements and current transmission for measurement of the total impedance, a driven shield electrode is used to focus current transmission to further restrict current flow through the scalp. Combined electrode unit 1900, driven shield combined electrode units 1910, 1920 and 1930 and reference electrode 1940 are placed on the scalp of a subject. The scalp impedance is measured by transmitting current from the inner electrode to the peripheral electrode of the combined electrode unit 1900 as shown by current path 1970, and measuring the resulting voltage drop as shown at 1980. Current is also transmitted from the center electrode of the driven shield combined electrode unit 1910 to the reference electrode 1940. Due to the forced isopotential areas of the driven shield combined electrode units, current is focused and restricted to some degree from flowing tangentially to the scalp as shown by the single dotted line representing current flow 1950 through the scalp, as compared to the double dotted line representing the primary current flow 1960 through the scalp, skull and neural tissue. The scalp impedance measurements collected from the combined electrode unit can then be subtracted from the impedance measurements obtained from the driven shield combined electrode units.

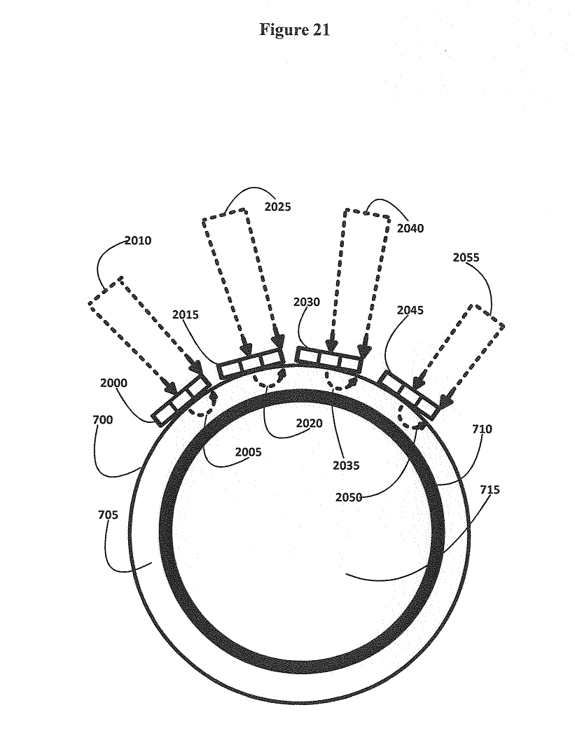

FIG. 21 shows a further embodiment of the scalp measurements described in FIG. 13. Multiple combined electrode units can be placed on the scalp to provide measurements of scalp impedance over multiple regions. In this case combined electrode units 2000, 2015, 2030, and 2045 are placed at different sites on the scalp layer 700. Currents 2005, 2020, 2035 and 2050 are transmitted from the center to the peripheral electrodes of the combined electrode units 2000, 2015, 2030, and 2045 respectively. Scalp impedance measurements can then be obtained from measuring the voltage drops 2010, 2025, 2040, and 2055. An array of combined electrode units covering the scalp could provide impedance measurements of the entire scalp layer. This could prove valuable for applications such as electrical impedance tomography where knowledge of scalp impedance is important for calculating an accurate image of the brain conductivity.

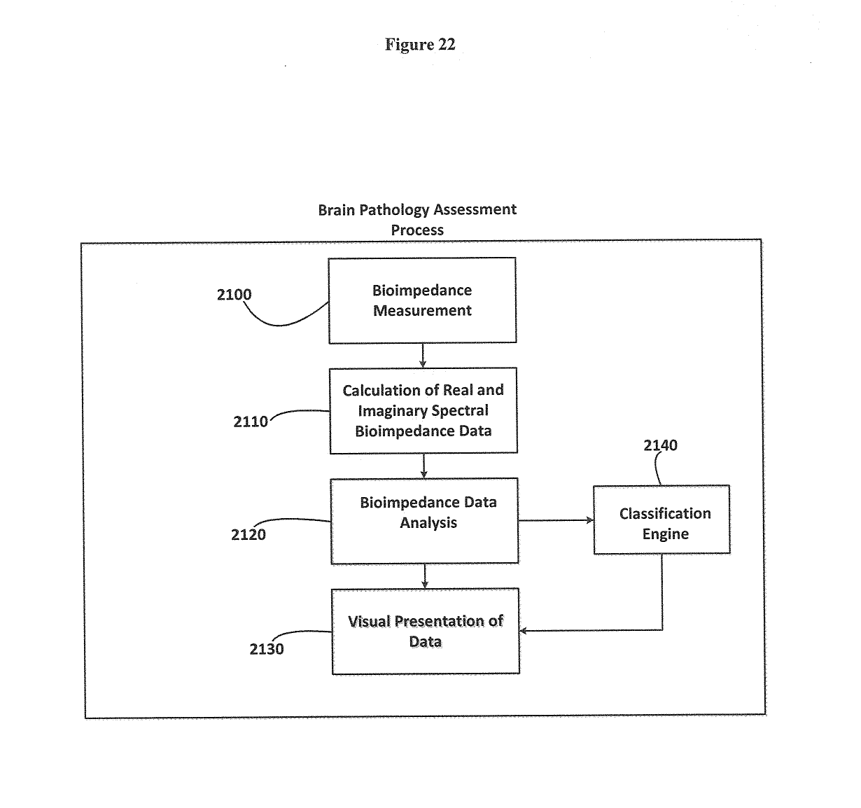

FIG. 22 shows the process of measuring, analyzing and post-processing impedance measurements. The first step is the impedance measurement step 2100 that may be obtained in any of the earlier described methods. The information is then processed in step 2110, which may include calculation of the real and imaginary components of bioimpedance known as the resistance and reactance over the frequency spectrum of the current and other information such as phase, admittance and capacitance. This information is then passed into step 2120 for further analysis. Further analysis may include cerebral blood flow, and plotting the impedance, the resistance and the reactance vs. frequency spectrum. The results of the analysis is further transmitted to step 2130 for visual display of the data. This data may then be analyzed by a trained human operator for comparison with other bioimpedance data gathered experimentally or collected from the published scientific literature for the purpose of identifying normal or abnormal spectra characteristic of neural tissue. This process bypasses any form of automated classification. Alternatively the results of the data analysis in step 2120 can be passed into a classification engine step 2140 for the purpose of analyzing and classifying the impedance data as characteristic of normal or abnormal neural tissue. The classification can then be transmitted into step 2130 for the purpose of graphical display.

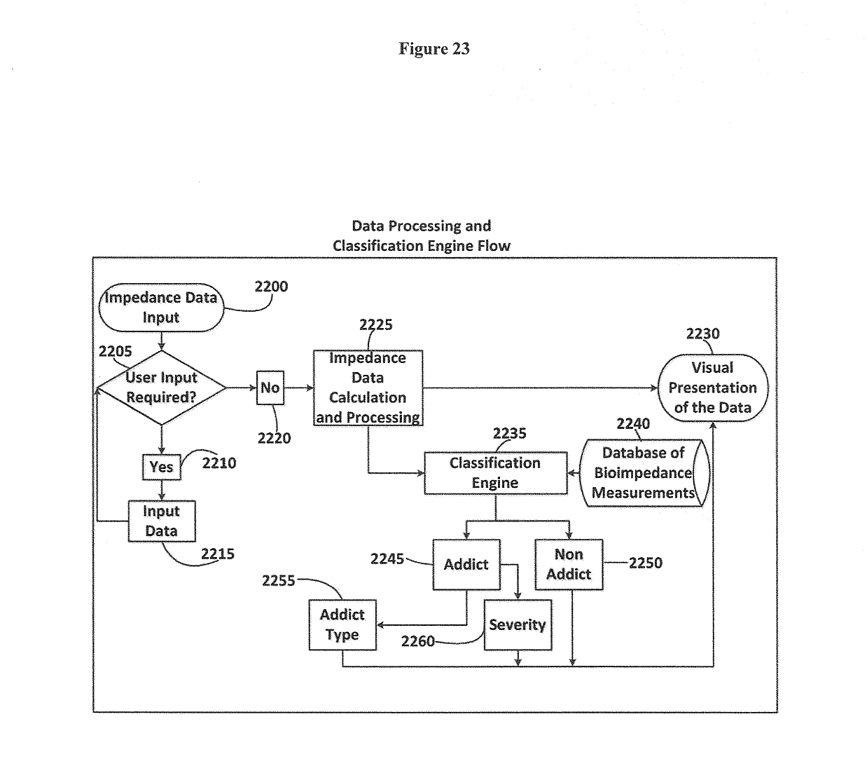

FIG. 23 provides further detail regarding the process of measuring, analysis, post-processing and classifying impedance measurements of the classification engine process. The described process begins with the input of impedance data such as the real and imaginary components of the measured impedance in step 2200. At step 2205, the user is requested to enter input if applicable. For example, cranial dimensions may be required to calculate the total intracranial fluid in the brain. If manually inputted data is required in step 2210, the process flow continues to step 2215 where the user enters the required data. The process flow then loops back to step 2205 to check if further user data is required. Once all the necessary user data has been inputted into the system, the input loop is exited through step 2220 and the impedance information is inputted into step 2225 for further data calculation and processing. This analysis may include calculation of total fluid in the intracranial cavity that can be approximated by V=CC²/R where V is the bioelectrical volume in square centimeters per ohm, CC is the cranial circumference and R is resistance in ohms (Grasso et al. 2002), cerebral blood flow, and plotting the impedance and/or the resistance and/or the reactance vs. frequency spectrum. Tissue impedance can be described through a model based analysis through extraction of Cole parameters as described in (Cole 1940). The information may bypass the classification engine (step 2235) and be inputted into step 2230 for graphical display of the data for the purpose of analysis and evaluation by a trained operator to determine if the impedance data are characteristic of an abnormal brain state. Alternatively the results of the impedance data analysis in step 2225 can be transferred to a classification engine in step 2235. The classification engine may analyze and classify the impedance data as characteristic of normal neural tissue or pathological neural tissue. The classifier may operate using the principle of artificial neural networks, support vector machines, or any other classifying paradigms. The classifying engine may compare plotted spectra of the impedance measurements with normal and abnormal plotted spectra of impedance measurements stored in an external database 2240. A pathological state may be identified and the degree of pathology may be quantified by indexing to a predetermined scale. In the example presented in FIG. 23 the classification engine will determine whether the impedance measurements are characteristic of an addict, whereby the results will be transmitted to step 2245. Alternatively, the impedance measurements will be classified as characteristic of a healthy non-addicted individual and results will be transmitted to step 2250 and then transmitted into step 2230 for visual presentation of the data for further review of a trained operator. If the classifying engine determines that the measured impedance data is characteristic of an addict as shown in 2245, further processing may occur, for example the classifying engine may determine the addiction type, passing the results of the data analysis to step 2255. In addition the severity of the addiction may be quantified by indexing to a predetermined scale at step 2260. The results from 2255 and 2260 may be further transmitted to step 2230 for visual presentation of the data by a trained operator. This process flow may also be used for the diagnosis of other pathologies besides for addiction including but not limited to stroke, subdural hematoma, and brain degenerative diseases. It is to be understood that method presented is an example of a preferred embodiment but is not presented in the limiting sense as other embodiments may be implemented.

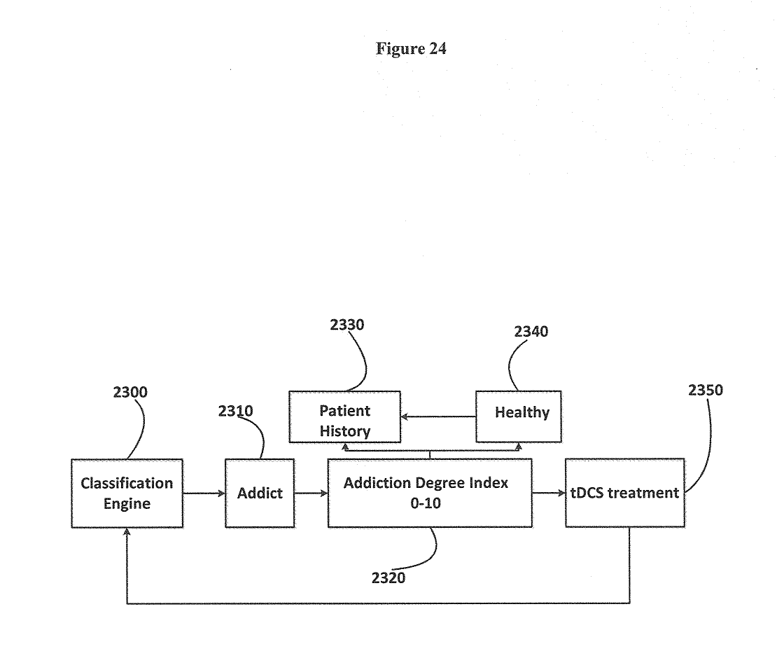

FIG. 24 shows a method of using the classification engine as part of an iterative loop with a treatment such as transcranial direct current stimulation. The method allows analysis of the effect of repeated treatments and the recording of any improvement in the pathological state if any exists. The classification engine 2300 first determines if the measured impedance data is characteristic of an addict as shown in 2310. Further analysis by the classification engine may quantify the degree of addiction by indexing to a predetermined scale shown in 2320 on a scale from 0-10. This index of severity may be transmitted to the patient history in 2330. After the addiction severity has been quantified, treatment such as transcranial direct current stimulation may be administered at targeted regions of the brain symbolized by step 2350. Following treatment the process may be repeated as shown by the connection of 2350 to the classification engine 2300. For each iteration of the process, a reassessment of the state of addiction will be obtained from the classification engine. In the event that the impedance data will be classified as characteristic of a healthy non-addict or 0 on the index scale, the subject will recorded as being healthy in step 2340 and the results will be stored in the patient history 2330. This process flow may also be used for the diagnosis and treatment of other pathologies besides for addiction. This description is not meant in the limiting sense but is one possible embodiment.

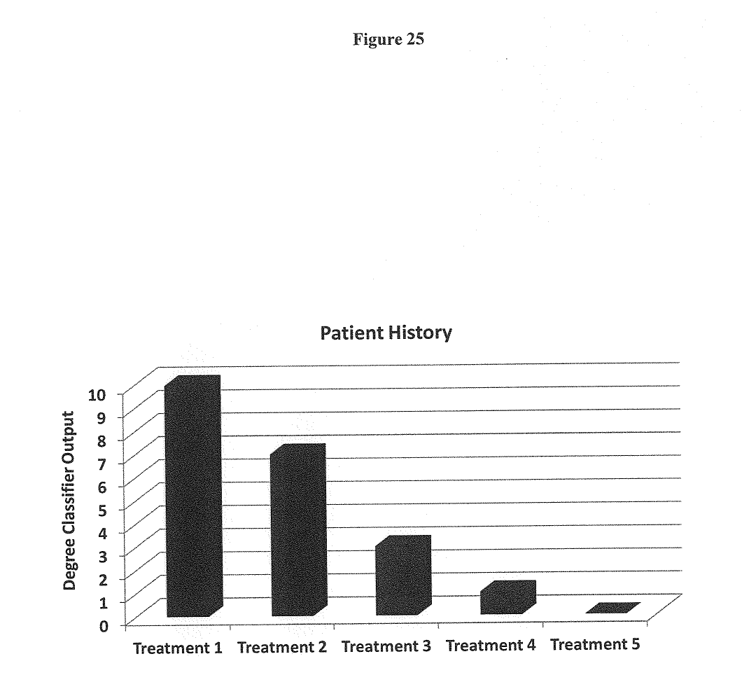

FIG. 25 shows an example of the classification engine output tracking the effects of the index of addiction severity vs. the number of transcranial direct current stimulation treatments. This illustration shows that repeated transcranial direct stimulation causes a reduction of addiction severity thereby showing a method of tracking the recovery of neural tissue from the pathological state of addiction. These processes may be used for other pathological conditions such as cancer or stroke, addiction is presented as one of the possible embodiments. In addition the transcranial direct current treatment described is only an example not meant in a limiting sense, and other techniques may be used in the treatment of addiction such as transcranial magnetic stimulation (TMS), drug based therapies such as methadone maintenance and cognitive behavioral therapy (CBT).

Although the invention has been described above by reference to certain embodiments of the invention, the invention is not limited to the embodiments described above. Modifications and variations of the embodiments described above will occur to those skilled in the art in light of the above teachings. Moreover, with respect to the above description, it is to be understood that the optimum dimensional relationships for the component members of the present invention may include variations in size, material, shape, form, funding and manner of operation.

We claim:

1. A method for determining brain state comprising the steps of:
a) attaching at least 2 first electrodes to the scalp of a subject at a separation;
b) transmitting an electrical current between the at least 2 first electrodes wherein the current has one or more frequencies within a predetermined frequency spectrum; and
c) measuring a first impedance between the at least 2 first electrodes to provide a resulting impedance.

2. The method of claim 1 further comprising the step of:
d) comparing the resulting impedance to pre-stored impedance measurements of the scalp for normal and pathological state.

3. The method of claim 1 wherein the at least 2 first electrodes are positioned in a combined electrode unit having a center electrode and a peripheral electrode having a separation between wherein the separation is less than a maximum scalp thickness further comprising the step of:
d) comparing the resulting impedance to pre-stored impedance measurements of the scalp for normal and pathological state.

4. The method of claim 1 wherein scalp impedance measurements are measured over a plurality of points on the scalp using more than one combined electrode units further comprising the step of:
d) comparing the resulting impedance to pre-stored impedance measurements of the scalp for normal and pathological state.

5. The method of claim 1 wherein the separation between the at least 2 first electrodes is less than maximum scalp thickness, the method further comprising the steps of:
d) attaching a reference electrode to the scalp of a subject wherein the separation between the reference electrode and at least one of the 2 first electrodes is at least maximum scalp thickness;
e) transmitting a electrical current between the at least one of the 2 first electrodes and the reference electrode having a separation at least maximum scalp thickness wherein the current has one or more frequencies within a predetermined frequency spectrum;
f) measuring a second impedance between the at least one of the 2 first electrodes and the reference electrode having a separation at least maximum scalp thickness;
g) subtracting the first impedance from the second impedance to provide a resulting impedance; and
h) comparing the resulting impedance to pre-stored impedance measurements for normal and pathological state.

6. The method of claim 5 wherein the at least 2 first electrodes are positioned in a combined electrode unit having a center electrode and a peripheral electrode having a separation between wherein the separation is less than a maximum scalp thickness and wherein the second impedance is measured between the center electrode of the combined electrode unit and the reference electrode.

7. The method according to claim 1, further comprising the steps of:
a) continually evaluating bioimpedance measurements for existence and degree of abnormality;
b) storing the information in the subject history; and
c) treating the subject with a neuromodulatory technique.

8. The method of claim 1 wherein the at least 2 first electrodes are positioned in a combined electrode unit having a center electrode and a peripheral electrode having a separation between wherein the separation is less than a maximum scalp thickness method further comprising:
d) attaching at least a reference electrode to the scalp of a subject;
e) attaching at least a first voltage electrode and second voltage electrode to the scalp of the subject where the separation between the voltage electrodes and the at least a third electrode is at least the maximum scalp thickness
f) transmitting a predetermined electrical current between one first electrode comprised of a center electrode of the combined electrode unit and the reference electrode wherein the current has one or more frequencies within a predetermined frequency spectrum;
g) measuring a second impedance between the first voltage electrode and the second voltage electrodes;
h) subtracting the first impedance from the second impedance to provide a resulting impedance; and
i) comparing the resulting impedance to pre-stored impedance measurements for normal and pathological state.

9. The method of claim 1 wherein the at least 2 first electrodes are positioned in a combined electrode unit having a center electrode and a peripheral electrode having a separation between wherein the separation is less than a maximum scalp thickness method further comprising:
d) attaching at least a current electrode to the scalp of a subject;
e) attaching at least a reference electrode to the scalp of a subject;
f) attaching at least a first voltage electrode and second voltage electrode to the scalp of the subject where the separation between the voltage electrodes and the at least a current electrode is at least the maximum scalp thickness;
g) transmitting a predetermined electrical current between the current electrode and the reference electrode wherein the current has one or more frequencies within a predetermined frequency spectrum;
h) measuring a second impedance between the first voltage electrode and the second voltage electrodes;
i) subtracting the first impedance from the second impedance to provide a resulting impedance; and
j) comparing the resulting impedance to pre-stored impedance measurements for normal and pathological state.

10. A method for determining brain state comprising the steps of:
a) attaching at least 2 first electrodes to the scalp of a subject at a separation;
b) attaching at least 2 second electrodes to the scalp of the subject;
c) transmitting a predetermined electrical current between the at least 2 first electrodes wherein the current has one or more frequencies within a predetermined frequency spectrum;
d) measuring a first impedance between the at least 2 second electrodes to provide a resulting impedance.

11. The method of claim 10 wherein the at least 2 first electrodes are comprised of a current electrode and a reference electrode, wherein the separation between the at least 2 first electrodes is at least maximum scalp thickness and wherein the 2 second electrodes consist of the current electrode and the center electrode of a combined electrode unit wherein a potential measured at the center electrode is forced onto the peripheral electrode by an operational amplifier, method further comprising:
d) comparing the resulting impedance measurements to pre-stored impedance measurements for normal and pathological state.

12. The method of claim 10 wherein the at least 2 first electrodes are comprised of a center electrode of a driven shield combined electrode unit and a reference electrode, wherein the predetermined distance between the at least 2 first electrodes is at least maximum scalp thickness and wherein the at least 2 second electrodes are comprised of 2 center electrodes of 2 driven shield combined electrode units method further comprising the step of:
d) comparing the resulting impedance measurements to pre-stored impedance measurements for normal and pathological state.

13. The method of claim 10 wherein the at least 2 first electrodes are comprised of a current electrode and a reference electrode, wherein the predetermined distance between the 2 first electrodes is at least maximum scalp thickness and wherein the at least 2 second electrodes are comprised of 2 center electrodes of 2 driven shield combined electrode unit method further comprising the step of:
d) comparing the resulting impedance measurements to pre-stored impedance measurements for normal and pathological state.

14. The method of claim 10 wherein the at least 2 first electrodes are comprised of a center electrode of a driven shield combined electrode unit and a reference electrode, wherein the predetermined distance between the at least 2 first electrodes is at least maximum scalp thickness and wherein the at least 2 second electrodes are comprised of 2 center electrodes of 2 driven shield combined electrode units method further comprising the step of:
d) comparing the resulting impedance measurements to pre-stored impedance measurements for normal and pathological state.

15. The method according to claim 10, further comprising the steps of:
a) continually evaluating bioimpedance measurements for existence and degree of abnormality;
b) storing the information in the subject history; and
c) treating the subject with a neuromodulatory technique.

16. The method of claim 1 wherein the at least 2 first electrodes are comprised of a center and peripheral electrode of a combined electrode unit wherein the predetermined distance between the at least 2 first electrodes is at least maximum scalp thickness method further comprising the steps of:
d) attaching at least 1 driven shield combined electrode unit to the scalp of the subject;
e) transmitting a predetermined electrical current between the center electrode of at least 1 combined electrode unit and the reference electrode, wherein the current has one or more frequencies within a predetermined frequency spectrum;
f) measuring a second impedance between 2 center electrodes comprised of 1 center electrode of the combined electrode unit and 1 center electrode of the driven shield combined electrode unit;
g) subtracting the first impedance from the second impedance to provide a resulting impedance measurement; and
h) comparing the resulting impedance measurements to pre-stored impedance measurements for normal and pathological state.

17. The method of claim 1 wherein the at least 2 first electrodes are comprised of a center and peripheral electrode of a combined electrode unit wherein the predetermined distance between the at least 2 first electrodes is at least maximum scalp thickness method further comprising the steps of:
d) attaching at least 2 driven shield combined electrode units to the scalp of the subject;
e) attaching at least 1 reference electrode to the scalp of the subject;
f) transmitting a predetermined electrical current between the center electrode of at least 1 combined electrode unit and 1 reference electrode wherein the current has one or more frequencies within a predetermined frequency spectrum;
g) measuring the impedance between the center electrodes of the at least 2 driven shield combined electrode units;
h) subtracting the first impedance from the second impedance to provide a resulting impedance measurement; and
i) comparing the resulting impedance measurements to pre-stored impedance measurements for normal and pathological state.

18. The method of claim 1 wherein the at least 2 first electrodes are comprised of a center and peripheral electrode of a combined electrode unit wherein the predetermined distance between the at least 2 first electrodes is at least maximum scalp thickness method further comprising the steps of:
d) attaching at least 3 driven shield combined electrode units to the scalp of the subject;
e) attaching at least 1 reference electrode to the scalp of the subject;
f) transmitting a predetermined electrical current between the center electrode of at least 1 driven shield combined electrode unit and 1 reference electrode wherein the current has one or more frequencies within a predetermined frequency spectrum;
g) measuring the impedance between the center electrodes of the at least 2 driven shield combined electrode units;
h) subtracting the first impedance from the second impedance to provide a resulting impedance measurement; and
i) comparing the resulting impedance measurements to pre-stored impedance measurements for normal and pathological state.

19. A combined electrode unit for determining scalp state comprising:
1) an inner electrode having a central conductive area;
2) a surrounding non-conductive insulating area; and
3) an outer electrode consisting of a peripheral conductive area
wherein a separation between an edge of the central conductive area and the peripheral conductive area is less than maximum scalp thickness.

20. The electrode according to claim 19 wherein the voltage measured at the inner electrode is forced on the peripheral conductive area by an operational amplifier.
